(19) United States
(12) Patent Application Publication
(10) Pub. No.: US 2006/0034776 A1
(43) Pub. Date: Feb. 16, 2006

(54) Inhalable medicaments containing a new anticholinergic, corticosteroids, and betamimetics

(75) Inventors: Michael P. Pieper, Biberach (DE); Michel Pairet, Biberach (DE)

Correspondence Address: MICHAEL P. MORRIS;BOEHRINGER INGELHEIM CORPORATION, 900 RIDGEBURY ROAD, P. O. BOX 368, RIDGEFIELD, CT, 06877-0368, US

(73) Assignee: Boehringer Ingelheim International GmbH, Ingelheim (DE)

(21) Appl. No.: 11/182,382

(22) Filed: Jul. 15, 2005

(30) Foreign Application Priority Data
Aug. 10, 2004 (DE) 10 2004 038 886
Nov. 3, 2004 (DE) 10 2004 053 023

(51) Int. Cl. A61L 9/04; A61K 31/573; A61K 31/4745

(52) U.S. Cl. 424/45; 514/171; 514/291

(57) ABSTRACT

A pharmaceutical formulation comprising: (a) at least one anticholinergic of formula 1 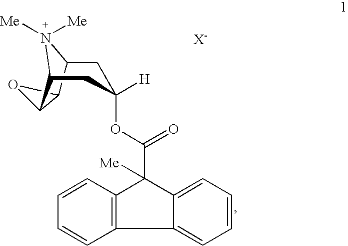
wherein X⁻ is an anion with a single negative charge; (b) at least one corticosteroid (2); and (c) at least one betamimetic (3), and the enantiomers, mixtures of the enantiomers, racemates, solvates, hydrates, or physiologically acceptable acid addition salts thereof, processes for preparing them and their use in the treatment of respiratory diseases.

### RELATED APPLICATIONS

This application claims priority to German Application No. 10 2004 038 886.5, filed Aug. 10, 2004, and German Application No. 10 2004 053 023.8, filed Nov. 3, 2004, each of which is hereby incorporated by reference in its entirety.

### FIELD OF THE INVENTION

The present invention relates to novel pharmaceutical compositions based on a new anticholinergic, corticosteroids, and betamimetics, processes for preparing them, and their use in the treatment of respiratory diseases.

### DESCRIPTION OF THE INVENTION

The present invention relates to novel pharmaceutical compositions for inhalation based on a new anticholinergic, corticosteroids, and betamimetics, processes for preparing them, and their use in the treatment of respiratory diseases.

Surprisingly, an unexpectedly beneficial therapeutic effect can be observed in the treatment of inflammatory or obstructive respiratory diseases when a new anticholinergic is used in conjunction with one or more corticosteroids and in conjunction with one or more betamimetics.

The effects mentioned above may be observed both when the three active substances are administered simultaneously in a single active substance formulation and when they are administered successively in separate formulations. According to the invention, it is preferable to administer the active substance ingredients simultaneously in a single formulation. The pharmaceutical compositions according to the invention are preferably administered by inhalation according to the invention.

Within the scope of the present invention the anticholinergics used are the salts of formula 1 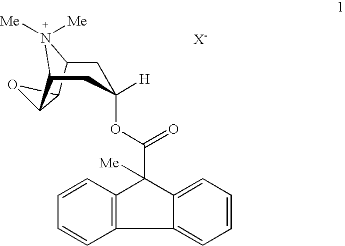
wherein:
X⁻ denotes an anion with a single negative charge, preferably an anion selected from the group consisting of chloride, bromide, iodide, sulfate, phosphate, methanesulfonate, nitrate, maleate, acetate, citrate, fumarate, tartrate, oxalate, succinate, benzoate, and p-toluenesulfonate.

Preferably, the salts of formula 1 are used wherein:
X⁻ denotes an anion with a single negative charge selected from the group consisting of chloride, bromide, 4-toluenesulfonate, and methanesulfonate, preferably bromide.

Most preferably, the salts of formula 1 are used wherein:
X⁻ denotes an anion with a single negative charge selected from the group consisting of chloride, bromide and methanesulfonate, preferably bromide.

Particularly preferred according to the invention is the salt of formula 1 wherein X⁻ denotes bromide.

The salts of formula 1 are known from International Patent Application WO 03/064419 (corresponding to U.S. Pat. Nos. 6,790,856 and 6,815,452, which are hereby incorporated by reference). Any reference to the salts of formula 1 includes a reference to any hydrates and solvates thereof which may be obtained.

Within the scope of the present patent application, an explicit reference to the pharmacologically active cation of formula 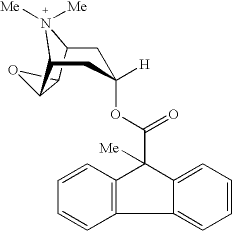
can be recognized by the use of the designation 1′. Any reference to compounds 1 naturally includes a reference to the cation 1′.

Within the scope of the present invention, the word corticosteroids (hereinafter 2 denotes compounds selected from among flunisolide (2.1), beclomethasone (2.2), triamcinolone (2.3), budesonide (2.4), fluticasone (2.5), mometasone (2.6), ciclesonide (2.7), rofleponide (2.8), GW 215864 (2.9), KSR 592 (2.10), ST-126 (2.11), dexamethasone (2.12 ), etiprednol (2.13), loteprednol (2.14), EPI-177 (2.15), and PLD-177 (2.16). Preferably, compound 2 is selected from among flunisolide (2.1), beclomethasone (2.2), triamcinolone (2.3), budesonide (2.4), fluticasone (2.5), mometasone (2.6), ciclesonide (2.7), etiprednol (2.13), EPI-177 (2.15), and dexamethasone (2.12). Particularly preferably the compound 2 is selected from among budesonide (2.4), fluticasone (2.5), mometasone (2.6), and ciclesonide (27), while mometasone (2.6) and ciclesonide (2.7) are of particular importance according to the invention. In some cases, within the scope of the present patent application, the term steroids 2 may also be used on its own instead of the word corticosteroids 2.

Any reference to steroids 2 within the scope of the present invention includes a reference to salts or derivatives 2′ which may be formed from the steroids. Examples of possible salts or derivatives 2′ include: sodium salts, sulfobenzoates, phosphates, isonicotinates, acetates, propionates, dihydrogen phosphates, palmitates, pivalates, or furoates. In some cases the compounds of formula 2 may also occur in the form of their hydrates.

Examples of betamimetics 3 according to the invention are preferably compounds selected from the group comprising albuterol (3.1), bambuterol (3.2), bitolterol (3.3), broxaterol (3.4), carbuterol (3.5), clenbuterol (3.6), fenoterol (3.7), formoterol (3.8), hexoprenaline (3.9), ibuterol (3.10), isoetharine (3.11), isoprenaline (3.12), levosalbutamol (3.13), mabuterol (3.14), meluadrine (3.15), metaproterenol (3.16), orciprenaline (3.17), pirbuterol (3.18), procaterol (3.19), reproterol (3.20), TD 3327 (3.21), ritodrin (3.22), salmeterol (3.23), salmefamol (3.24), soterenot (3.25), sulfonterol (3.26), tiaramide (3.27), terbutaline (3.28), tolubuterol (3.29), CHF-4226 (TA 2005 or carmoterol) (3.30), HOKU-81 (3.31), KUL-1248 (3.32), 3-(4-{6-[2-hydroxy-2-(4-hydroxy-3-hydroxymethylphenyl)ethylamino]hexyloxy}butyl)benzenesulfonamide (3.33), 5-[2-(5,6-diethylindan-2-ylamino)-1-hydroxyethyl]-8-hydroxy-1H-quinolin-2-one (3.34), 4-hydroxy- 7-[2-{[2-{[3-(2-phenylethoxy)propyl]sulfonyl}ethyl]amino}ethyl]-2(3H)-benzothiazolone (3.35), 1-(2-fluoro-4-hydroxyphenyl)-2-[4-(1-benzimidazolyl)-2-methyl-2-butylamino]ethanol (3.36), 1-[3-(4-methoxybenzylamino)-4-hydroxyphenyl]-2-[4-(1-benzimidazolyl)-2-methyl-2-butylamino]ethanol (3.37), 1-[2H-5-hydroxy-3-oxo-4H-1,4-benzoxazin-8-yl]-2-[3-(4-N,N-dimethylaminophenyl)-2-methyl-2-propylamino]ethanol (3.38), 1-[2H-5-hydroxy-3-oxo-4H-1,4-benzoxazin-8-yl]-2-[3-(4-methoxyphenyl)-2-methyl-2-propylamino]ethanol (3.39), 1-[2H-5-hydroxy-3-oxo-4H-1,4-benzoxazin-8-yl]-2-[3-(4-n-butyloxyphenyl)-2-methyl-2-propylamino]ethanol (3.40), 1-[2H-5-hydroxy-3-oxo-4H-1,4-benzoxazin-8-yl]-2-{4-[3-(4-methoxyphenyl)-1,2,4-triazol-3-yl]-2-butylamino}ethanol (3.41), 5-hydroxy-8-(1-hydroxy-2-isopropylaminobutyl)-2H-1,4-benzoxazin-3-(4H)-one (3.42), 1-(4-amino-3-chloro-5-trifluoromethylphenyl)-2-tert-butylamino)ethanol (3.43), 1-(4-ethoxycarbonylamino-3-cyano-5-fluorophenyl)-2-(tert-butylamino)ethanol (3.44), and N-[2-hydroxy-5-(1-hydroxy-2-{2-[4-(2-hydroxy-2-phenylethylamino)phenyl]ethylamino}ethyl)phenyl]formamide (3.45), optionally in the form of the racemates, enantiomers, and diastereomers and optionally in the form of the physiologically acceptable acid addition salts and hydrates thereof.

Preferably, the β₂ agonists (betamimetics) 1 in the combinations according to the invention are selected from bambuterol (3.2), bitolterol (3.3), carbuterol (3.5), clenbuterol (3.6), fenoterol (3.7), formoterol (3.8), hexoprenaline (3.9), ibuterol (3.10), pirbuterol (3.18), procaterol (3.19), reproterol (3.20), TD 3327 (3.21), salmeterol (3.23), sulfonterol (3.26), terbutaline (3.28), tolubuterol (3.29), CHF-4226 (3.30), 3-(4-{6-[2-hydroxy-2-(4-hydroxy-3-hydroxymethylphenyl)ethylamino]hexyloxy}butyl)benzenesulfonamide (3.33), 5-[2-(5,6-diethylindan-2-ylamino)-1-hydroxyethyl]-8-hydroxy-1H-quinolin-2-one (3.34), 4-hydroxy-7-[2-{[2-{[3-(2-phenylethoxy)propyl]sulfonyl}ethyl]amino}ethyl]-2(3H)-benzothiazolone (3.35), 1-(2-fluoro-4-hydroxyphenyl)-2-[4-(1-benzimidazolyl)-2-methyl-2-butylamino]ethanol (3.36), 1-[3-(4-methoxybenzylamino)-4-hydroxyphenyl]-2-[4-(1-benzimidazolyl)-2-methyl-2-butylamino]ethanol (3.37), 1-[2H-5-hydroxy-3-oxo-4H-1,4-benzoxazin-8-yl]-2-[3-(4-N,N-dimethylaminophenyl)-2-methyl-2-propylamino]ethanol (3.38), 1-[2H-5-hydroxy-3-oxo-4H-1,4-benzoxazin-8-yl]-2-[3-(4-methoxyphenyl)-2-methyl-2-propylamino]ethanol (3.39), 1-[2H-5-hydroxy-3-oxo-4H-1,4-benzoxazin-8-yl]-2-[3-(4-n-butyloxyphenyl)-2-methyl-2-propylamino]ethanol (3.40), 1-[2H-5-hydroxy-3-oxo-4H-1,4-benzoxazin-8-yl]-2-{4-[3-(4-methoxyphenyl)-1,2,4-triazol-3-yl]-2-methyl-2-butylamino}ethanol (3.41), 5-hydroxy-8-(1-hydroxy-2-isopropylaminobutyl)-2H-1,4-benzoxazin-3-(4H)-one (3.42), 1-(4-amino-3-chloro-5-trifluoromethylphenyl)-2-tert-butylamino)ethanol (3.43), 1-(4-ethoxycarbonylamino-3-cyano-5-fluorophenyl)-2-(tert-butylamino)ethanol (3.44), and N-[2-hydroxy-5-(1-hydroxy-2-{2-[4-(2-hydroxy-2-phenylethylamino)phenyl]ethylamino}ethyl)phenyl]formamide (3.45), optionally in the form of the racemates, enantiomers, and diastereomers and optionally in the form of the physiologically acceptable acid addition salts and hydrates thereof.

Also particularly preferred within the scope of the combinations according to the invention are those betamimetics 1 which are selected from the group comprising 3.7, 3.8, 3.23, 3.30, 3.33, 3.34, 3.37, 3.38, 3.39, 3.40, 3.41, and 3.45, optionally in the form of the racemates, enantiomers, and diastereomers and optionally in the form of the physiologically acceptable acid addition salts and hydrates thereof.

Of the abovementioned betamimetics the following compounds are of particular significance according to the invention: 3.8, 3.23, 3.30, 3.33, 3.34, and 3.45, optionally in the form of the racemates, enantiomers, and diastereomers and optionally in the form of the physiologically acceptable acid addition salts and hydrates thereof.

Any reference to the term betamimetics 3 also includes a reference to the relevant enantiomers or mixtures thereof. Compounds which are in the R configuration at the C—OH— carbon are particularly preferred. For example, any reference to the particularly preferred compounds 3 according to the invention, the salts of salmeterol and formoterol, also includes the relevant enantiomeric salts of R-salmeterol, S-salmeterol, R,R-formoterol, S,S-formoterol, R,S-formoterol, S,R-formoterol, and the mixtures thereof, while the enantiomeric salts of R-salmeterol and R,R-formoterol are of particular importance. The compounds 3 may also be present according to the invention in the form of the hydrates or solvates thereof.

Within the scope of the present invention any reference to compounds 3 is to be understood as being a reference to physiologically acceptable acid addition salts. By physiologically acceptable acid addition salts of the betamimetics 3 are meant according to the invention pharmaceutically acceptable salts which are selected from the salts of hydrochloric acid, hydrobromic acid, sulfuric acid, phosphoric acid, methanesulfonic acid, acetic acid, fumaric acid, succinic acid, lactic acid, citric acid, tartaric acid, 1-hydroxy-2-naphthalenecarboxylic acid, cinnamic acid, 4-phenylcinnamic acid, diflunisal, or maleic acid. If desired, mixtures of the abovementioned acids may be used to prepare the salts 3.

According to the invention the salts of the betamimetics 3 selected from among the hydrochloride, hydrobromide, sulfate, phosphate, fumarate, methanesulfonate, 4-phenylcinnamate, diflunisal, and xinafoate are preferred. Particularly preferably, in the case of salmeterol, the salts of 3 are selected from those salts which have a solubility in water of 0.1 mg/mL or less, preferably 0.05 mg/mL or less, most preferably 0.04 mg/mL or less. In this context xinafoate, 4-phenylcinnamate, and diflunisal are mentioned as particularly preferred salts of salmeterol. Particularly preferred salts 3 of salmeterol have a solubility in water of 0.035 mg/mL or less, such as, for example, 4-phenylcinnamate or diflunisal.

Particularly preferably, in the case of formoterol, the salts of 3 are selected from the hydrochloride, sulfate and fumarate, of which the hydrochloride and fumarate are particularly preferred. Of exceptional importance according to the invention is formoterol fumarate, for example, preferably formoterol fumarate dihydrate or formoterol hemifumarate, preferably in the form of its monohydrate.

Within the scope of the present invention the betamimetics 3 are optionally also referred to as sympathomimetics or β₂-receptor agonists (β₂-agonists). All these names can be regarded as equivalent within the scope of the present invention.

The pharmaceutical combinations of 1, 2, and 3 according to the invention are preferably administered by inhalation. Suitable inhalable powders packed into suitable capsules (inhalettes) may be administered using suitable powder inhalers. Accordingly, in one aspect, the present invention relates to a pharmaceutical composition which contains a combination of 1, 2, and 3.

In another aspect the present invention relates to a pharmaceutical composition which contains one or more salts 1, one or more compounds 2, and one or more compounds 3, optionally in the form of their solvates or hydrates. The active substances may either be combined in a single preparation or contained in two or three separate formulations. Pharmaceutical compositions which contain the active substances 1, 2, and 3 in a single preparation are preferred according to the invention.

In another aspect the present invention relates to a pharmaceutical composition which contains, in addition to therapeutically effective quantities of 1, 2, and 3, a pharmaceutically acceptable excipient. In another aspect the present invention relates to a pharmaceutical composition which does not contain any pharmaceutically acceptable excipient in addition to therapeutically effective quantities of 1, 2, and 3.

The present invention also relates to the use of 1, 2, and 3 for preparing a pharmaceutical composition containing therapeutically effective quantities of 1, 2, and 3 for treating inflammatory and/or obstructive diseases of the respiratory tract, particularly asthma and/or chronic obstructive pulmonary disease (COPD), by simultaneous or successive administration. In addition the pharmaceutical combinations according to the invention may be used to prepare a drug for treating fibrotic pulmonary diseases such as cystic fibrosis or allergic alveolitis (farmer's lung), for example, by simultaneous or successive administration. The combinations of active substances according to the invention will not be used only if treatment with one of the pharmaceutically active ingredients is contraindicated.

The present invention also relates to the simultaneous or successive use of therapeutically effective doses of the combination of the above pharmaceutical compositions 1, 2, and 3 for treating inflammatory or obstructive diseases of the respiratory tract, particularly asthma and/or chronic obstructive pulmonary disease (COPD), provided that treatment with steroids or betamimetics is not contraindicated from a therapeutic point of view, by simultaneous or successive administration. The invention further relates to the simultaneous or successive use of therapeutically effective doses of the combination of the above pharmaceutical compositions 1, 2, and 3 for treating cystic fibrosis or allergic alveolitis (farmer's lung), for example.

In the active substance combinations of 1, 2, and 3 according to the invention, ingredients 1, 2, and 3 may be present in the form of their enantiomers, mixtures of enantiomers or in the form of racemates. For example, the pharmaceutical compositions according to the invention contain the active substances 1, 2, and 3 according to the invention in amounts such that a single administration corresponds to a dosage of the combination of active substances 1, 2, and 3 of 1 to 10000 μg, preferably from 10 to 2000 μg.

The proportions in which the active substances 1, 2, and 3 may be used in the active substance combinations according to the invention are variable. Active substances 1, 2, and 3 may possibly be present in the form of their solvates or hydrates. Depending on the choice of the compounds 1, 2, and 3, the weight ratios which may be used within the scope of the present invention vary on the basis of the different molecular weights of the various compounds and their different potencies. As a rule, the pharmaceutical combinations according to the invention may contain compounds 1′ and 2 in ratios by weight ranging from 1:250 to 250:1, preferably from 1:150 to 150:1. In the particularly preferred pharmaceutical combinations which contain in addition to 1′ a compound selected from among budesonide, fluticasone, mometasone, ST-126, and ciclesonide as steroid 2, the weight ratios of 1′ to 2 are most preferably in a range from about 1:40 to 40:1, more preferably in the range from 1:30 to 30:1.

For example, without restricting the scope of the invention thereto, preferred combinations of 1 and 2 according to the invention may contain the cation 1′ and one of the abovementioned preferred steroids 2 in the following proportions by weight: 1:40; 1:39; 1:38, 1:37; 1:36; 1:35; 1:34; 1:33; 1:32; 1:31; 1:30; 1:29; 1:28; 1:27; 1:26; 1:25; 1:24; 1:23; 1:22; 1:21; 1:20; 1:19; 1:18; 1:17; 1:16; 1:15; 1:14; 1:13; 1:12; 1:11; 1:10; 1:9; 1:8; 1:7; 1:6; 1:5; 1:4; 1:3; 1:2; 1:1; 2:1; 3:1; 4:1; 5:1; 6:1; 7:1; 8:1; 9:1; 10:1; 11:1; 12:1; 13:1; 14:1; 15:1; 16:1; 17:1; 18:1; 19:1; and 20:1.

The pharmaceutical compositions according to the invention containing the combinations of 1 and 2 are normally administered so that 1′ and 2 are present together in doses of 5 to 5000 μg, preferably from 10 to 5000 μg, more preferably from 15 to 4500 μg, even more preferably from 20 to 4000 μg, preferably according to the invention from 30 to 3500 μg, preferably from 40 to 3000 μg, preferably from 50 to 2500 μg, preferably from 40 to 2250 μg, more preferably from 50 to 2000 μg per single dose. For example, combinations of 1 and 2 according to the invention contain a quantity of 1′ and steroid 2 such that the total dosage per single dose is about 35 μg, 45 μg, 50 μg, 55 μg, 60 μg, 65 μg, 70 μg, 75 μg, 80 μg, 85 μg, 90 μg, 95 μg, 100 μg, 105 μg, 110 μg, 115 μg, 120 μg, 125 μg, 130 μg, 153 μg, 140 μg, 145 μg, 150 μg, 155 μg, 160 μg, 165 μg, 170 μg, 175 μg, 180 μg, 185 μg, 190 μg, 195 μg, 200 μg, 205 μg, 210 μg, 215 μg, 220 μg, 225 μg, 230 μg, 235 μg, 240 μg, 245 μg, 250 μg, 255 μg, 260 μg, 265 μg, 270 μg, 275 μg, 280 μg, 285 μg, 290 μg, 295 μg, 300 μg, 305 μg, 310 μg, 315 μg, 320 μg, 325 μg, 330 μg, 335 μg, 340 μg, 345 μg, 350 μg, 355 μg, 360 μg, 365 μg, 370 μg, 375 μg, 380 μg, 385 μg, 390 μg, 395 μg, 400 μg, 405 μg, 410 μg, 415 μg, 420 μg, 425 μg, 430 μg, 435 μg, 440 μg, 445 μg, 450 μg, 455 μg, 460 μg, 465 μg, 470 μg, 475 μg, 480 μg, 485 μg, 490 μg, 495 μg, 500 μg, 505 μg, 510 μg, 515 μg, 520 μg, 525 μg, 530 μg, 535 μg, 540 μg, 545 μg, 550 μg, 555 μg, 560 μg, 565 μg, 570 μg, 575 μg, 580 μg, 585 μg, 590 μg, 595 μg, 600 μg, 605 μg, 610 μg, 610 μg, 615 μg, 620 μg, 625 μg, 630 μg, 635 μg, 640 μg, 645 μg, 650 μg, 655 μg, 660 μg, 665 μg, 670 μg, 675 μg, 680 μg, 685 μg, 690 μg, 695 μg, 700 μg, 705 μg, 710 μg, 715 μg, 720 μg, 725 μg, 730 μg, 735 μg, 740 μg, 745 μg, 750 μg, 755 μg, 760 μg, 765 μg, 770 μg, 775 μg, 780 μg, 785 μg, 790 μg, 795 μg, 800 μg, 805 μg, 810 μg, 815 μg, 820 μg, 825 μg, 830 μg, 835 μg, 840 μg, 845 μg, 850 μg, 855 μg, 860 μg, 865 μg, 870 μg, 875 μg, 880 μg, 885 μg, 890 μg, 895 μg, 900 μg, 905 μg, 910 μg, 915 μg, 920 μg, 925 μg, 930 μg, 935 μg, 940 μg, 945 μg, 950 μg, 955 μg, 960 μg, 965 μg, 970 μg, 975 μg, 980 μg, 985 μg, 990 μg, 995 μg, 1000 μg, 1005 μg, 1010 μg, 1015 μg, 1020 μg, 1025 μg, 1030 μg, 1035 μg, 1040 μg, 1045 μg, 1050 μg, 1055 μg, 1060 μg, 1065 μg, 1070 μg, 1075 μg, 1080 μg, 1085 μg, 1090 μg, 1095 μg, 1100 μg, 1105 μg, 1110 μg, 1115 μg, 1120 μg, 1125 μg, 1130 μg, 1135 μg, 1140 μg, 1145 μg, 1150 μg, 1155 μg, 1160 μg, 1165 μg, 1170 μg, 1175 μg, 1180 μg, 1185 μg, 1190 μg, 1195 μg, 1200 μg, 1205 μg, 1210 μg, 1215 μg, 1220 μg, 1225 μg, 1230 μg, 1235 μg, 1240 μg, 1245 μg, 1250 μg, 1255 μg, 1260 μg, 1265 μg, 1270 μg, 1275 μg, 1280 μg, 1285 μg, 1290 μg, 1295 μg, 1300 μg, 1305 μg, 1310 μg, 1315 μg, 1320 μg, 1325 μg, 1330 μg, 1335 μg, 1340 μg, 1345 μg, 1350 μg, 1355 μg, 1360 μg, 1365 μg, 1370 μg, 1375 μg, 1380 μg, 1385 μg, 1390 μg, 1395 μg, 1400 μg, 1405 μg, 1410 μg, 1415 μg, 1420 μg, 1425 μg, 1430 μg, 1435 μg, 1440 μg, 1445 μg, 1450 μg, 1455 μg, 1460 μg, 1465 μg, 1470 μg, 1475 μg, 1480 μg, 1485 μg, 1490 μg, 1495 μg, 1500 μg, 1505 μg, 1510 μg, 1515 μg, 1520 μg, 1525 μg, 1530 μg, 1535 μg, 1540 μg, 1545 μg, 1550 μg, 1555 μg, 1560 μg, 1565 μg, 1570 μg, 1575 μg, 1580 μg, 1585 μg, 1590 μg, 1595 μg, 1600 μg, 1605 μg, 1610 μg, 1615 μg, 1620 μg, 1625 μg, 1630 μg, 1635 μg, 1640 μg, 1645 μg, 1650 μg, 1655 μg, 1660 μg, 1665 μg, 1670 μg, 1675 μg, 1680 μg, 1685 μg, 1690 μg, 1695 μg, 1700 μg, 1705 μg, 1710 μg, 1715 μg, 1720 μg, 1725 μg, 1730 μg, 1735 μg, 1740 μg, 1745 μg, 1750 μg, 1755 μg, 1760 μg, 1765 μg, 1770 μg, 1775 μg, 1780 μg, 1785 μg, 1790 μg, 1795 μg, 1800 μg, 1805 μg, 1810 μg, 1815 μg, 1820 μg, 1825 μg, 1830 μg, 1835 μg, 1840 μg, 1845 μg, 1850 μg, 1855 μg, 1860 μg, 1865 μg, 1870 μg, 1875 μg, 1880 μg, 1885 μg, 1890 μg, 1895 μg, 1900 μg, 1905 μg, 1910 μg, 1915 μg, 1920 μg, 1925 μg, 1930 μg, 1935 μg, 1940 μg, 1945 μg, 1950 μg, 1955 μg, 1960 μg, 1965 μg, 1970 μg, 1975 μg, 1980 μg, 1985 μg, 1990 μg, 1995 μg, 2000 μg, or the like It is clear to the skilled person that these proposed dosages per single dose are not to be regarded as being restricted to the numerical values explicitly mentioned. Fluctuations of around ±2.5 μg, particularly fluctuations in the decimal range, are also covered as will be apparent to anyone skilled in the art. In these dosage ranges the active substances 1′ and 2 may be present in the weight ratios described above.

For example and without restricting the scope of the invention thereto, the combinations of 1 and 2 according to the invention may contain an amount of cation 1′ and steroid 2 such that each single dose contains 2.5 μg of 1′ and 25 μg of 2, 2.5 μg of 1′ and 50 μg of 2, 2.5 μg of 1′ and 100 μg of 2, 2.5 μg of 1′ and 150 μg of 2, 2.5 μg of 1′ and 200 μg of 2, 2.5 μg, of 1′ and 250 μg of 2, 2.5 μg of 1′ and 300 μg of 2, 2.5 μg of 1′ and 320 μg of 2, 2.5 μg of 1′ and 350 μg of 2, 2.5 μg of 1′ and 400 μg of 2, 2.5 μg of 1′ and 500 μg of 2, 2.5 μg of 1′ and 600 μg of 2, 2.5 μg of 1′ and 700 μg of 2, 2.5 μg of 1′ and 800 μg of 2,2.5 μg of 1′ and 1000 μg of 2, 7.5 μg of 1′ and 25 μg of 2, 7.5 μg of 1′ and 50 μg of 2, 7.5 μg of 1′ and 100 μg of 2, 7.5 μg of 1′ and 150 μg of 2, 7.5 μg of 1′ and 200 μg of 2, 7.5 μg of 1′ and 250 μg of 2, 7.5 μg of 1′ and 300 μg of 2, 7.5 μg of 1′ and 320 μg of 2, 7.5 μg of 1′ and 350 μg of 2, 7.5 μg of 1′ and 400 μg of 2, 7.5 μg of 1′ and 500 μg of 2, 7.5 μg of 1′ and 600 μg of 2, 7.5 μg of 1′ and 700 μg of 2, 7.5 μg of 1′ and 800 μg of 2, 7.5 μg of 1′ and 1000 μg of 2, 15 μg of 1′ and 25 μg of 2, 15 μg of 1′ and 50 μg of 2, 15 μg of 1′ and 100 μg of 2, 15 μg of 1′ and 150 μg of 2, 15 μg of 1′ and 200 μg of 2, 15 μg of 1′ and 250 g of 2, 15 μg of 1′ and 300 μg of 2, 15 μg of 1′ and 320 μg of 2, 15 μg of 1′ and 350 μg of 2, 15 μg of 1′ and 400 μg of 2, 15 μg of 1′ and 500 μg of 2, 15 μg of 1′ and 600 μg of 2, 15 μg of 1′ and 700 μg of 2, 15 μg of 1′ and 800 μg of 2, 15 μg of 1′ and 1000 μg of 2, 30.0 μg of 1′ and 25 μg of 2, 30.0 μg of 1′ and 50 μg of 2, 30.0 μg of 1′ and 100 μg of 2, 30.0 μg of 1′ and 150 μg of 2, 30.0 μg of 1′ and 200 μg of 2, 30.0 μg of 1′ and 250 μg of 2, 30.0 μg of 1′ and 300 μg of 2, 30.0 μg of 1′ and 320 μg of 2, 30.0 μg of 1′ and 350 μg of 2, 30.0 μg of 1′ and 400 μg of 2, 30.0 μg of 1′ and 500 μg of 2, 30.0 μg of 1′ and 600 μg of 2, 30.0 μg of 1′ and 700 μg of 2, 30.0 μg of 1′ and 800 μg of 2, 30.0 μg of 1′ and 1000 μg of 2, 60.0 μg of 1′ and 25 μg of 2, 60.0 μg of 1′ and 50 μg of 2, 60.0 μg of 1′ and 100 μg of 2, 60.0 μg of 1′ and 150 μg of 2, 60.0 μg of 1′ and 200 μg of 2, 60.0 μg of 1′ and 250 μg of 2, 60.0 μg of 1′ and 300 μg of 2, 60.0 μg of 1′ and 320 μg of 2, 60.0 μg of 1′ and 350 μg of 2, 60.0 μg of 1′ and 400 μg of 2, 60.0 μg of 1′ and 500 μg of 2, 60.0 μg of 1′ and 600 μg of 2, 60.0 μg of 1′ and 700 μg of 2, 60.0 μg of 1′ and 800 μg of 2, 60.0 μg of 1′ and 1000 μg of 2, 90.0 μg of 1′ and 25 μg of 2, 90.0 μg of 1′ and 50 μg of 2, 90.0 μg of 1′ and 100 μg of 2, 90.0 μg of 1′ and 150 μg of 2, 90.0 μg of 1′ and 200 μg of 2, 90.0 μg of 1′ and 250 μg of 2, 90.0 μg of 1′ and 300 μg of 2, 90.0 μg of 1′ and 320 μg of 2, 90.0 μg of 1′ and 350 μg of 2, 90.0 μg of 1′ and 400 μg of 2, 90.0 μg of 1′ and 500 μg of 2, 90.0 μg of 1′ and 600 μg of 2, 90.0 μg of 1′ and 700 μg of 2, 90.0 μg of 1′ and 800 μg of 2, and 90.0 μg of 1′ and 1000 μg of 2.

At the same time the ratio of 1 to 3 may be 1:500 to 30:1, preferably from 1:400 to 20:1, more preferably from 1:300 to 10:1, still more preferably from 1:200 to 5:1, more preferably from 1:100 to 2:1. In the case of formoterol, for example, the active substance combinations according to the invention may contain 1′ and 3′ in ratios by weight which are, for example, in the range from about 1:50 to 300:1, preferably 1:20 to 200:1, preferably 1:10 to 150:1, more preferably from 1:5 to 100:1.

For example and without restricting the scope of the invention thereto, preferred combinations of 1 and 3 according to the invention contain the pharmacologically active cation 1′ and formoterol 3 in the following ratios by weight: 1:80, 1:79, 1:78, 1:77, 1:76, 1:75, 1:74, 1:73, 1:72, 1:71, 1:70, 1:69, 1:68, 1:67, 1:66, 1:65, 1:64, 1:63, 1:62, 1:61, 1:60, 1:59, 1:58, 1:57, 1:56, 1:55, 1:54, 1:53, 1:52, 1:51, 1:50, 1:49, 1:48, 1:47, 1:46, 1:45, 1:44, 1:43, 1:42, 1:41, 1:40, 1:39, 1:38, 1:37, 1:36, 1:35, 1:34, 1:33, 1:32, 1:31, 1:30, 1:29, 1:28, 1:27, 1:26, 1:25, 1:24, 1:23, 1:22, 1:21, 1:20, 1:19, 1:18, 1:17, 1:16, 1:15, 1:14, 1:13, 1:12, 1:11, 1:10, 1:10, 1:9, 1:8, 1:7, 1:6, 1:5, 1:4, 1:3, 1:2, 1:1, 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, 21:1, 22:1, 23:1, 24:1, 25:1, 26:1, 27:1, 28:1, 29:1, 30:1, 31:1, 32:1, 33:1, 34:1, 35:1, 36:1, 37:1, 38:1, 39:1, 40:1, 41:1, 42:1, 43:1, 44:1, 45:1, 46:1, 47:1, 48:1, 49:1, 50:1, 51:1, 52:1, 53:1, 54:1, 55:1, 56:1, 57:1, 58:1, 59:1, 60:1, 61:1, 62:1, 63:1, 64:1, 65:1, 66:1, 67:1, 68:1, 69:1, 70:1, 71:1, 72:1, 73:1, 74:1, 75:1, 76:1, 77:1, 78:1, 79:1, and 80:1.

The pharmaceutical compositions according to the invention containing the combinations of 1 and 3 are normally used so that the pharmacologically active cation 1′ and formoterol 3 are present together in doses from 0.05 to 2500 μg, preferably from 0.5 to 1250 μg, more preferably from 1.0 to 1000 μg, still more preferably from 5 to 750 μg, preferably according to the invention from 10 to 500 μg, preferably from 15 to 250 μg.

For example, the combinations of 1 and 3 according to the invention contain an amount of cation 1′ and formoterol 3 such that the total dosage per single dose is about 0.5 μg, 1 μg, 5 μg, 10 μg, 15 μg, 20 μg, 25 μg, 30 μg, 35 μg, 45 μg, 50 μg, 55 μg, 60 μg, 65 μg, 70 μg, 75 μg, 80 μg, 85 μg, 90 μg, 95 μg, 100 μg, 105 μg, 110 μg, 115 μg, 120 μg, 125 μg, 130 μg, 135 μg, 140 μg, 145 μg, 150 μg, 155 μg, 160 μg, 165 μg, 170 μg, 175 μg, 180 μg, 185 μg, 190 μg, 195 μg, 200 μg, 205 μg, 210 μg, 215 μg, 220 μg, 225 μg, 230 μg, 235 μg, 240 μg, 245 μg, 250 μg, 255 μg, 260 μg, 265 μg, 270 μg, 275 μg, 280 μg, 285 μg, 290 μg, 295 μg, 300 μg, 305 μg, 310 μg, 315 μg, 320 μg, 325 μg, 330 μg, 335 μg, 340 μg, 345 μg, 350 μg, 355 μg, 360 μg, 365 μg, 370 μg, 375 μg, 380 μg, 385 μg, 390 μg, 395 μg, 400 μg, 405 μg, 410 μg, 415 μg, 420 μg, 425 μg, 430 μg, 435 μg, 440 μg, 445 μg, 450 μg, or similar.

It is clear to the skilled man that these proposed dosages per single dose are not to be regarded as being restricted to the numerical values explicitly mentioned. Fluctuations of around ±2.5 μg, particularly fluctuations in the decimal range, are also covered as will be apparent to anyone skilled in the art. In these dosage ranges the active substances 1′ and 3 are present in the weight ratios described above.

For example and without restricting the scope of the invention thereto, the combinations of 1 and 3 according to the invention contain an amount of cation 1′ and formoterol 3 (based on free base) such that they contain, per single dose, for example, 2.5 μg of 1′ and 2.5 μg of 3, 2.5 μg of 1′ and 4.9 μg of 3, 2.5 μg of 1′ and 9.8 μg of 3, 2.5 μg of 1′ and 14.7 μg of 3, 2.5 μg of 1′ and 19.6 μg of 3, 2.5 μg of 1′ and 24.4 μg of 3, 7.5 μg of 1′ and 2.5 μg of 3, 7.5 μg of 1′ and 4.9 μg of 3, 7.5 μg of 1′ and 9.8 μg of 3, 7.5 μg of 1′ and 14.7 μg of 3, 7.5 μg of 1′ and 19.6 μg of 3, 7.5 μg of 1′ and 24.4 μg of 3, 15.0 μg of 1′ and 2.5 μg of 3, 15.0 μg of 1′ and4.9 μg of 3, 15.0 μg of 1′ and 9.8 μg of 3, 15.0 μg of 1′ and 14.7 g of 3, 15.0 μg of 1′ and 19.6 μg of 3, 15.0 μg of 1′ and 24.4 μg of 3, 30.0 μg of 1′ and 2.5 μg of 3, 30.0 μg of 1′ and 4.9 μg of 3, 30.0 μg of 1′ and 9.8 μg of 3, 30.0 μg of 1′ and 14.7 μg of 3, 30.0 μg of 1′ and 19.6 μg of 3, 30.0 μg of 1′ and 24.4 μg of 3, 60.0 μg of 1′ and 2.5 μg of 3, 60.0 μg of 1′ and 4.9 μg of 3, 60.0 μg of 1′ and 9.8 μg of 3, 60.0 μg of 1′ and 14.7μg of 3, 60.0 μg of 1′ and 19.6 μg of 3, 60.0 μg of 1′ and 24.4 μg of 3, 90.0 μg of 1′ and 2.5 μg of 3, 90.0 μg of 1′ and 4.9 μg of 3, 90.0 μg of 1′ and 9.8 μg of 3, 90.0 μg of 1′ and 14.7 μg of 3, 90.0 μg of 1′ and 19.6 μg of 3, and 90.0 μg of 1′ and 24.4 μg of 3.

In the case of salmeterol, for example, the active substance combinations according to the invention may contain 1′ and 3 in ratios by weight which are in the range from about 1:30 to 400:1, preferably 1:25 to 200:1, preferably 1:20 to 100:1, more preferably from 1:15 to 50:1, for example.

For example, and without restricting the scope of the invention thereto, the preferred combinations of 1 and 3 according to the invention may contain the cation 1′ and salmeterol 3 (based on free base) in the following ratios by weight: 1:15, 1:14, 1:13, 1:12, 1:11, 1:10, 1:9, 1:8, 1:7, 1:6, 1:5, 1:4, 1:3, 1:2, 1:1, 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, 21:1, 22:1, 23:1, 24:1, 25:1, 26:1, 27:1, 28:1, 29:1, 30:1, 31:1, 32:1, 33:1, 34:1, and 35:1.

The pharmaceutical compositions according to the invention containing the combinations of 1 and 3 are normally used so that the cation 1′ and salmeterol 3 (based on free base) are present together in doses from 5 to 5000 μg, preferably from 10 to 2000 μg, more preferably from 15 to 1000 μg, still more preferably from 20 to 800 μg, preferably according to the invention from 30 to 750 μg, preferably from 40 to 700 μg.

For example, the combinations of 1 and 3 according to the invention contain an amount of 1′ and salmeterol 3 (based on free base) such that the total dosage per single dose is about 15 μg, 20 μg, 25 μg, 30 μg, 35 μg, 45 μg, 50 μg, 55 μg, 60 μg, 65 μg, 70 μg, 75 μg, 80 μg, 85 μg, 90 μg, 95 μg, 100 μg, 105 μg, 110 μg, 115 μg, 120 μg, 125 μg, 130 μg, 135 μg, 140 μg, 145 μg, 150 μg, 155μg, 160 μg, 165 μg, 170 μg, 175 μg, 180 μg, 185 μg, 190 μg, 195 μg, 200 μg, 205 μg, 210 μg, 215 μg, 220 μg, 225 μg, 230 μg, 235 μg, 240 μg, 245 μg, 250 μg, 255 μg, 260 μg, 265 μg, 270 μg, 275 μg, 280 μg, 285 μg, 290 μg, 295 μg, 300 μg, 305 μg, 310 μg, 315 μg, 320 μg, 325 μg, 330 μg, 335 μg, 340 μg, 345 μg, 350 μg, 355 μg, 360 μg, 365 μg, 370 μg, 375 μg, 380 μg, 385 μg, 390 μg, 395 μg, 400 μg, 405 μg, 410 μg, 415 μg, 420 μg, 425 μg, 430 μg, 435 μg, 440 μg, 445 μg, 450 μg, or similar. It is clear to the skilled man that these proposed dosages per single dose are not to be regarded as being restricted to the numerical values explicitly mentioned. Fluctuations of around +2.5 μg, particularly fluctuations in the decimal range, are also covered as will be apparent to anyone skilled in the art. In these dosage ranges the active substances 1′ and 3 are present in the weight ratios described above.

For example and without restricting the scope of the invention thereto, the combinations of 1 and 3 according to the invention may contain an amount of cation 1′ and salmeterol 3 (based on free base) such that they contain, per single dose, for example 2.5 μg of 1′ and 12.5 μg of 3, 2.5 μg of 1′ and 25 μg of 3, 2.5 μg of 1′ and 50 μg of 3, 2.5 μg of 1′ and 75 μg of 3, 2.5 μg of 1′ and 100 μg of 3, 2.5 μg of 1′ and 200 μg of 3, 7.5 μg of 1′ and 12.5 μg of 3, 7.5 μg of 1′ and 25 μg of 3, 7.5 μg of 1′ and 50 μg of 3, 7.5 μg of 1′ and 75 μg of 3, 7.5 μg of 1′ and 100 μg of 3, 7.5 μg of 1′ and 200 μg of 3, 15.0 μg of 1′ and 12.5 μg of 3, 15.0 μg of 1′ and 25 μg of 3, 15.0 μg of 1′ and 50 μg of 3, 15.0 μg of 1′ and 75 μg of 3, 15.0 μg of 1′ and 100 μg of 3, 15.0 μg of 1′ and 200 μg of 3, 30.0 μg of 1′ and 12.5 μg of 3, 30.0 μg of 1′ and 25 μg of 3, 30.0 μg of 1′ and 50 μg of 3, 30.0 μg of 1′ and 75 μg of 3, 30.0 μg of 1′ and 100 μg of 3, 30.0 μg of 1′ and 200 μg of 3, 60.0 μg of 1′ and 12.5 μg of 3, 60.0 μg of 1′ and 25 μg of 3, 60.0 μg of 1′ and 50 μg of 3, 60.0 μg of 1′ and 75 μg of 3, 60.0 μg of 1′ and 100 μg of 3, 60.0 μg of 1′ and 200 μg of 3, 490.0 μg of 1′ and 12.5 μg of 3, 490.0 μg of 1′ and 25 μg of 3, 490.0 μg of 1′ and 50 μg of 3, 490.0 μg of 1′ and 75 μg of 3, 490.0 μg of 1′ and 100 μg of 3, 490.0 μg of 1′ and 200 μg of 3.

The corresponding amounts of the salts of formula 1 and the corresponding amounts of any hydrates and/or solvates of the salts 1 which may exist are easily calculated by the skilled man from the amounts of 1′ specified above, taking into account the mass of the anion X⁻, and optionally taking into account the mass of the hydrate water and/or solvate molecules. Similarly, the corresponding amounts of the salts 2 or 3 and the corresponding amounts of any existing hydrates and/or solvates of the salts 2 or 3 are easily calculated by the skilled man from the amounts of 2′ or 3 specified above.

Pharmaceutical combinations which are preferred according to the invention are selected from the group comprising: 1, 2.1, and 3.7; 1, 2.1, and 3.8; 1, 2.1, and 3.23; 1, 2.1, and 3.30; 1, 2.1, and 3.33; 1, 2.1, and 3.34; 1, 2.1, and 3.37; 1, 2.1, and 3.38; 1, 2.1, and 3.39; 1, 2.1, and 3.40; 1, 2.1, and 3.41; 1, 2.1, and 3.45; 1, 2.2, and 3.7; 1, 2.2, and 3.8; 1, 2.2, and 3.23; 1, 2.2, and 3.30; 1, 2.2, and 3.33; 1, 2.2, and 3.34; 1, 2.2, and 3.37; 1, 2.2, and 3.38; 1, 2.2, and 3.39; 1, 2.2, and 3.40; 1, 2.2, and 3.41; 1, 2.2, and 3.45; 1, 2.3, and 3.7; 1, 2.3, and 3.8; 1, 2.3, and3;1, 3,and 3.30; 1, 2.3, and 3.33; 1, 2.3, and 3.34; 1, 2.3, and 3.37; 1, 2.3, and 3.38; 1, 2.3, and 3.39; 1, 2.3, and 3.40; 1, 2.3, and 3.41; 1, 2.3, and 3.45; 1, 2.4, and 3.7; 1, 2.4, and 3.8; 1, 2.4, and 3.23; 1, 2.4, and 3.30; 1, 2.4, and 3.33; 1, 2.4, and 3.34; 1, 2.4, and 3.37; 1, 2.4, and 3.38; 1, 2.4, and 3.39; 1, 2.4, and 3.40; 1, 2.4, and 3.41; 1, 2.4, and 3.45; 1, 2.5, and 3.7; 1, 2.5, and 3.8; 1, 2.5, and 3.23; 1, 2.5, and 3.30; 1, 2.5, and 3.33; 1, 2.5, and 3.34; 1, 2.5, and 3.37; 1, 2.5, and 3.38; 1, 2.5, and 3.39; 1, 2.5, and 3.40; 1, 2.5, and 3.41; 1, 2.5, and 3.45; 1, 2.6, and 3.7; 1, 2.6, and 3.8; 1, 2.6, and 3.23; 1, 2.6, and 3.30; 1, 2.6, and 3.33; 1, 2.6, and 3.34; 1, 2.6, and 3.37; 1, 2.6, and 3.38; 1, 2.6, and 3.39; 1, 2.6, and 3.40; 1, 2.6, and 3.41; 1, 2.6, and 3.45; 1, 2.7, and 3.7; 1, 2.7, and 3.8; 1, 2.7, and 3.23; 1, 2.7, and 3.30; 1, 2.7, and 3.33; 1, 2.7, and 3.34; 1, 2.7, and 3.37; 1, 2.7, and 3.38; 1, 2.7, and 3.39; 1, 2.7, and 3.40; 1, 2.7, and 3.41; 1, 2.7, and 3.45; 1, 2.12, and 3.7; 1, 2.12, and 3.8; 1, 2.12, and 3.23; 1, 2.12, and 3.30; 1, 2.12, and 3.33; 1, 2.12, and 3.34; 1, 2.12, and 3.37; 1, 2.12, and 3.38; 1, 2.12, and 3.39; 1, 2.12, and 3.40; 1, 2.12, and 3.41 or 1, 2.12, and 3.45. In the pharmaceutical combinations mentioned hereinbefore, 1 is particularly preferably in the form of the bromide.

The active substance combinations of 1, 2, and 3 according to the invention are preferably administered by inhalation. For this purpose, ingredients 1, 2, and 3 have to be made available in forms suitable for inhalation. Inhalable preparations include inhalable powders, propellant-containing metered-dose aerosols, or propellant-free inhalable solutions. Inhalable powders according to the invention containing the combination of active substances 1, 2, and 3 may consist of the active substances on their own or of a mixture of the active substances with physiologically acceptable excipients. Within the scope of the present invention the term carrier may optionally be used instead of the term excipient. Within the scope of the present invention, the term propellant-free inhalable solutions also includes concentrates or sterile inhalable solutions ready for use. The preparations according to the invention may contain the combination of active substances 1, 2, and 3 either together in one formulation or in two or three separate formulations. These formulations which may be used within the scope of the present invention are described in more detail in the next part of the specification.

A. Inhalable Powder

The inhalable powders according to the invention may contain 1, 2, and 3 either on their own or in admixture with suitable physiologically acceptable excipients.

If the active substances 1, 2, and 3 are present in admixture with physiologically acceptable excipients, the following physiologically acceptable excipients may be used to prepare these inhalable powders according to the invention: monosaccharides (e.g., glucose or arabinose), disaccharides (e.g., lactose, saccharose, or maltose), oligo- and polysaccharides (e.g., dextran), polyalcohols (e.g., sorbitol, mannitol, or xylitol), salts (e.g., sodium chloride or calcium carbonate) or mixtures of these excipients. Preferably, mono- or disaccharides are used, while the use of lactose or glucose is preferred, particularly, but not exclusively, in the form of their hydrates. For the purposes of the invention, lactose is the particularly preferred excipient, while lactose monohydrate is most particularly preferred.

Within the scope of the inhalable powders according to the invention the excipients have a maximum average particle size of up to 250 μm, preferably between 10 and 150 μm, most preferably between 15 and 80 μm. It may sometimes seem appropriate to add finer excipient fractions with an average particle size of 1 to 9 μm to the excipients mentioned above. These finer excipients are also selected from the group of possible excipients listed hereinbefore. Finally, in order to prepare the inhalable powders according to the invention, micronized active substance 1, 2, and 3, preferably with an average particle size of 0.5 to 10 μm, more preferably from 1 to 5 μm, is added to the excipient mixture. Processes for producing the inhalable powders according to the invention by grinding and micronizing and lastly mixing the ingredients together are known from the prior art. The inhalable powders according to the invention may be prepared and administered either in the form of a single powder mixture which contains both 1 and 2 and 3 or in the form of separate inhalable powders which contain only 1, 2, or 3.

The inhalable powders according to the invention may be administered using inhalers known from the prior art. Inhalable powders according to the invention which contain a physiologically acceptable excipient in addition to 1, 2, and 3 may be administered, for example, by means of inhalers which deliver a single dose from a supply using a measuring chamber as described in U.S. Pat. No. 4,570,630, or by other means as described in DE 36 25 685 A, each of which is incorporated by reference herein. Preferably, the inhalable powders according to the invention which contain physiologically acceptable excipients in addition to 1, 2, and 3 are packed into capsules (to produce so-called inhalettes) which are used in inhalers as described, for example, in WO 94/28958 (corresponding to U.S. Pat. No. 5,947,118, which is hereby incorporated by reference).

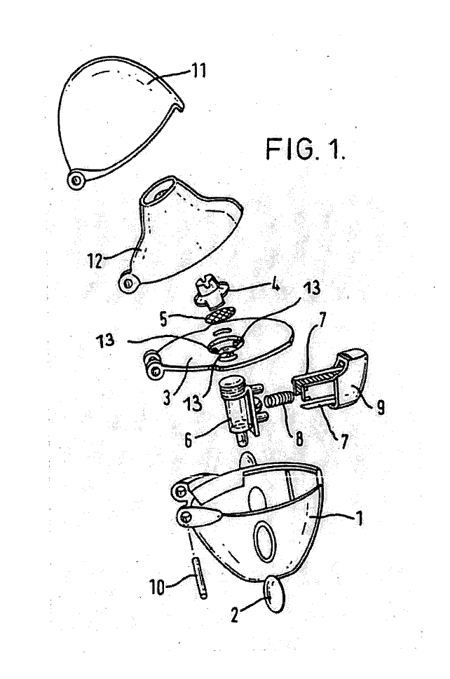

A particularly preferred inhaler for using the pharmaceutical combination according to the invention in inhalettes is shown in FIG. 1. This inhaler (sold under the trademark HANDIHALER®) for inhaling powdered pharmaceutical compositions from capsules is characterized by a housing 1 containing two windows 2, a deck 3 in which there are air inlet ports and which is provided with a screen 5 secured via a screen housing 4, an inhalation chamber 6 connected to the deck 3 on which there is a push button 9 provided with two sharpened pins 7 and movable counter to a spring 8, and a mouthpiece 12 which is connected to the housing 1, the deck 3 and a cover 11 via a spindle 10 to enable it to be flipped open or shut and air holes 13 for adjusting the flow resistance.

If the inhalable powders according to the invention are to be packed into capsules (inhalettes) for the preferred use described above, the quantities packed into each capsule should be 1 to 30 mg, preferably 3 to 20 mg, more particularly 5 to 10 mg of inhalable powder per capsule. These capsules contain, according to the invention, either together or separately, the doses of 1, 2, and 3 mentioned hereinbefore for each single dose.

B. Propellant Gas-Driven Inhalation Aerosols

Inhalation aerosols containing propellant gas according to the invention may contain substances 1, 2, and 3 dissolved in the propellant gas or in dispersed form. 1, 2, and 3 may be present in separate formulations or in a single preparation, in which 1, 2, and 3 are either each dissolved, dispersed or only one or two of the components is or are dissolved and the other or others is or are dispersed. The propellant gases which may be used to prepare the inhalation aerosols according to the invention are known from the prior art. Suitable propellant gases are selected from among hydrocarbons such as n-propane, n-butane or isobutane and halohydrocarbons such as fluorinated derivatives of methane, ethane, propane, butane, cyclopropane or cyclobutane. The propellant gases mentioned above may be used on their own or in mixtures thereof. Particularly preferred propellant gases are halogenated alkane derivatives selected from TG134a, TG227, and mixtures thereof.

The propellant-driven inhalation aerosols according to the invention may also contain other ingredients such as cosolvents, stabilizers, surfactants, antioxidants, lubricants, preservatives and pH adjusters. All these ingredients are known in the art.

The inhalation aerosols containing propellant gas according to the invention may contain up to 5 wt.-% of active substance 1, 2 and/or 3. Aerosols according to the invention contain, for example, 0.002 to 5 wt.-%, 0.01 to 3 wt.-%, 0.015 to 2 wt.-%, 0.1 to 2 wt.-%, 0.5 to 2 wt.-% or 0.5 to 1 wt.-% of active substance 1, 2, and/or 3.

If the active substances 1, 2, and/or 3 are present in dispersed form, the particles of active substance preferably have an average particle size of up to 10 μm, preferably from 0.1 to 5 μm, more preferably from 1 to 5 μm.

The propellant-driven inhalation aerosols according to the invention mentioned above may be administered using inhalers known in the art, such as metered dose inhalers (MDIs). Accordingly, in another aspect, the present invention relates to pharmaceutical compositions in the form of propellant gas-containing aerosols as hereinbefore described combined with one or more inhalers suitable for administering these aerosols. In addition, the present invention relates to inhalers which are characterized in that they contain the propellant gas-containing aerosols described above according to the invention. The present invention also relates to cartridges which are fitted with a suitable valve and can be used in a suitable inhaler and which contain one of the abovementioned propellant gas-containing inhalation aerosols according to the invention. Suitable cartridges and methods of filling these cartridges with the inhalable aerosols containing propellant gas according to the invention are known from the prior art.

C. Propellant-Free Inhalable Solutions or Suspensions

It is particularly preferred to use the active substance combination according to the invention in the form of propellant-free inhalable solutions and suspensions. The solvent used may be an aqueous or alcoholic, preferably an ethanolic solution. The solvent may be water on its own or a mixture of water and ethanol. The relative proportion of ethanol compared with water is not limited but the maximum is up to 70 percent by volume, more particularly up to 60 percent by volume and most preferably up to 30 percent by volume. The remainder of the volume is made up of water. The solutions or suspensions containing 1, 2, and 3, separately or together, are adjusted to a pH of 2 to 7, preferably 2 to 5, using suitable acids. The pH may be adjusted using acids selected from inorganic or organic acids. Examples of particularly suitable inorganic acids include hydrochloric acid, hydrobromic acid, nitric acid, sulfuric acid, and/or phosphoric acid. Examples of particularly suitable organic acids include ascorbic acid, citric acid, malic acid, tartaric acid, maleic acid, succinic acid, fumaric acid, acetic acid, formic acid, and/or propionic acid etc. Preferred inorganic acids are hydrochloric and sulfuric acids. It is also possible to use the acids which have already formed an acid addition salt with one of the active substances. Of the organic acids, ascorbic acid, fumaric acid and citric acid are preferred. If desired, mixtures of the above acids may be used, particularly in the case of acids which have other properties in addition to their acidifying qualities, e.g., as flavorings, antioxidants or complexing agents, such as citric acid or ascorbic acid, for example. According to the invention, it is particularly preferred to use hydrochloric acid to adjust the pH.

According to the invention, the addition of edetic acid (EDTA) or one of the known salts thereof, sodium edetate, as stabilizer or complexing agent is unnecessary in the present formulation. Other embodiments may contain this compound or these compounds. In a preferred embodiment the content based on sodium edetate is less than 100 mg/100 mL, preferably less than 50 mg/100 mL, more preferably less than 20 mg/100 mL. Generally, inhalable solutions in which the content of sodium edetate is from 0 to 10 mg/100 mL are preferred.

Cosolvents and/or other excipients may be added to the propellant-free inhalable solutions according to the invention. Preferred cosolvents are those which contain hydroxyl groups or other polar groups, e.g., alcohols—particularly isopropyl alcohol, glycols—particularly propyleneglycol, polyethyleneglycol, polypropylene glycol, glycol ether, glycerol, polyoxyethylene alcohols, and polyoxyethylene fatty acid esters. The terms excipients and additives in this context denote any pharmacologically acceptable substance which is not an active substance but which can be formulated with the active substance or substances in the physiologically suitable solvent in order to improve the qualitative properties of the active substance formulation. Preferably, these substances have no pharmacological effect or, in connection with the desired therapy, no appreciable or at least no undesirable pharmacological effect. The excipients and additives include, for example, surfactants such as soya lecithin, oleic acid, sorbitan esters, such as polysorbates, polyvinylpyrrolidone, other stabilizers, complexing agents, antioxidants, and/or preservatives which guarantee or prolong the shelf life of the finished pharmaceutical formulation, flavorings, vitamins and/or other additives known in the art. The additives also include physiologically acceptable salts such as sodium chloride as isotonic agents.

The preferred excipients include antioxidants such as ascorbic acid, for example, provided that it has not already been used to adjust the pH, vitamin A, vitamin E, tocopherols, and similar vitamins and provitamins occurring in the human body.

Preservatives may be used to protect the formulation from contamination with pathogens. Suitable preservatives are those which are known in the art, particularly cetyl pyridinium chloride, benzalkonium chloride, or benzoic acid or benzoates such as sodium benzoate in the concentration known from the prior art. The preservatives mentioned above are preferably present in concentrations of up to 50 mg/100 mL, more preferably between 5 and 20 mg/100 mL.

Preferred formulations contain, in addition to the solvent water and the combination of active substances 1, 2 and 3, only benzalkonium chloride and sodium edetate. In another preferred embodiment, no sodium edetate is present.

The propellant-free inhalable solutions according to the invention are administered in particular using inhalers of the kind which are capable of nebulizing a small amount of a liquid formulation in the required therapeutic dose within a few seconds to produce an aerosol suitable for therapeutic inhalation. Within the scope of the present invention, preferred nebulizers are those in which a quantity of less than 100 μL, preferably less than 50 μL, more preferably between 20 and 30 μL of active substance solution can be nebulized in preferably one spray action to form an aerosol with an average particle size of less than 20 μm, preferably less than 10 μm, in such a way that the inhalable part of the aerosol corresponds to the therapeutically effective quantity.

An apparatus of this kind for propellant-free delivery of a metered quantity of a liquid pharmaceutical composition for inhalation is described for example in International Patent Application WO 91/14468 (corresponding to U.S. Pat. No. 5,497,944, which is hereby incorporated by reference) and also in WO 97/12687 (cf. in particular FIGS. 6a and 6b) (corresponding to U.S. Pat. No. 5,964,416, which is hereby incorporated by reference). The nebulizers (devices) described therein are sold under the trademark RESPIMAT®.

This RESPIMAT® nebulizer can advantageously be used to produce the inhalable aerosols according to the invention containing the combination of active substances 1, 2, and 3. Because of its cylindrical shape and handy size of less than 9 to 15 cm long and 2 to 4 cm wide, this device can be carried at all times by the patient. The nebulizer sprays a defined volume of pharmaceutical formulation through small nozzles at high pressures so as to produce inhalable aerosols.

The preferred atomizer essentially consists of an upper housing part, a pump housing, a nozzle, a locking mechanism, a spring housing, a spring and a storage container, characterized by:
a pump housing which is secured in the upper housing part and which comprises at one end a nozzle body with the nozzle or nozzle arrangement,
a hollow plunger with valve body,
a power takeoff flange in which the hollow plunger is secured and which is located in the upper housing part,
a locking mechanism situated in the upper housing part,
a spring housing with the spring contained therein, which is rotatably mounted on the upper housing part by means of a rotary bearing, and
a lower housing part which is fitted onto the spring housing in the axial direction.

The hollow plunger with valve body corresponds to a device disclosed in WO 97/12687. It projects partially into the cylinder of the pump housing and is axially movable within the cylinder. Reference is made in particular to FIGS. 1 to 4, especially FIG. 3, and the relevant parts of the description. The hollow plunger with valve body exerts a pressure of 5 to 60 MPa (about 50 to 600 bar), preferably 10 to 60 MPa (about 100 to 600 bar) on the fluid, the measured amount of active substance solution, at its high pressure end at the moment when the spring is actuated. Volumes of 10 to 50 microliters are preferred, while volumes of 10 to 20 microliters are particularly preferred and a volume of 15 microliters per spray is most particularly preferred.

The valve body is preferably mounted at the end of the hollow plunger facing the nozzle body.

The nozzle in the nozzle body is preferably microstructured, i.e., produced by microtechnology. Microstructured nozzle bodies are disclosed, for example, in WO 94/07607; reference is hereby made to the contents of this specification, particularly FIG. 1 therein and the associated description. WO 94/07607 is incorporated herein by reference in its entirety.

The nozzle body consists for example of two sheets of glass and/or silicon firmly joined together, at least one of which has one or more microstructured channels which connect the nozzle inlet end to the nozzle outlet end. At the nozzle outlet end there is at least one round or non-round opening 2 to 10 microns deep and 5 to 15 microns wide, the depth preferably being 4.5 to 6.5 microns while the length is preferably 7 to 9 microns.

In the case of a plurality of nozzle openings, preferably two, the directions of spraying of the nozzles in the nozzle body may extend parallel to one another or may be inclined relative to one another in the direction of the nozzle opening. In a nozzle body with at least two nozzle openings at the outlet end the directions of spraying may be at an angle of 20° to 160° to one another, preferably 60° to 150°, most preferably 80° to 100°. The nozzle openings are preferably arranged at a spacing of 10 to 200 microns, more preferably at a spacing of 10 to 100 microns, most preferably 30 to 70 microns. Spacings of 50 microns are most preferred. The directions of spraying will therefore meet in the vicinity of the nozzle openings.

The liquid pharmaceutical preparation strikes the nozzle body with an entry pressure of up to 600 bar, preferably 200 to 300 bar, and is atomized into an inhalable aerosol through the nozzle openings. The preferred particle or droplet sizes of the aerosol are up to 20 microns, preferably 3 to 10 microns.

The locking mechanism contains a spring, preferably a cylindrical helical compression spring, as a store for the mechanical energy. The spring acts on the power takeoff flange as an actuating member the movement of which is determined by the position of a locking member. The travel of the power takeoff flange is precisely limited by an upper and lower stop. The spring is preferably biased, via a power step-up gear, e.g., a helical thrust gear, by an external torque which is produced when the upper housing part is rotated counter to the spring housing in the lower housing part. In this case, the upper housing part and the power takeoff flange have a single or multiple V-shaped gear.

The locking member with engaging locking surfaces is arranged in a ring around the power takeoff flange. It consists, for example, of a ring of plastic or metal which is inherently radially elastically deformable. The ring is arranged in a plane at right angles to the atomizer axis. After the biasing of the spring, the locking surfaces of the locking member move into the path of the power takeoff flange and prevent the spring from relaxing. The locking member is actuated by means of a button. The actuating button is connected or coupled to the locking member. In order to actuate the locking mechanism, the actuating button is moved parallel to the annular plane, preferably into the atomizer; this causes the deformable ring to deform in the annular plane. Details of the construction of the locking mechanism are given in WO 97/20590.

The lower housing part is pushed axially over the spring housing and covers the mounting, the drive of the spindle and the storage container for the fluid.

When the atomizer is actuated the upper housing part is rotated relative to the lower housing part, the lower housing part taking the spring housing with it. The spring is thereby compressed and biased by means of the helical thrust gear and the locking mechanism engages automatically. The angle of rotation is preferably a whole-number fraction of 360°, e.g., 180°. At the same time as the spring is biased, the power takeoff part in the upper housing part is moved along by a given distance, the hollow plunger is withdrawn inside the cylinder in the pump housing, as a result of which some of the fluid is sucked out of the storage container and into the high pressure chamber in front of the nozzle.

If desired, a number of exchangeable storage containers which contain the fluid to be atomized may be pushed into the atomizer one after another and used in succession. The storage container contains the aqueous aerosol preparation according to the invention.

The atomizing process is initiated by pressing gently on the actuating button. As a result, the locking mechanism opens up the path for the power takeoff member. The biased spring pushes the plunger into the cylinder of the pump housing. The fluid leaves the nozzle of the atomizer in atomized form.

Further details of the construction are disclosed in PCT applications WO 97/12683 corresponding to U.S. Pat. No. 6,176,442, which is hereby incorporated by reference) and WO 97/20590 (corresponding to U.S. Pat. No. 6,176,442, which is hereby incorporated by reference), to which reference is hereby made.

The components of the atomizer (nebulizer) are made of a material which is suitable for its purpose. The housing of the atomizer and, if its operation permits, other parts as well, are preferably made of plastics, e.g., by injection molding. For medicinal purposes, physiologically safe materials are used.

FIGS. 6a/b of WO 97/12687 to which reference is hereby made, together with the associated passages of description, show the nebulizer (RESPIMAT®) which can advantageously be used for inhaling the aqueous aerosol preparations according to the invention. FIG. 6a shows a longitudinal section through the atomizer with the spring biased while FIG. 6b shows a longitudinal section through the atomizer with the spring relaxed.

The upper housing part (51) contains the pump housing (52) on the end of which is mounted the holder (53) for the atomizer nozzle. In the holder is the nozzle body (54) and a filter (55). The hollow plunger (57) fixed in the power takeoff flange (56) of the locking mechanism projects partially into the cylinder of the pump housing. At its end the hollow plunger carries the valve body (58). The hollow plunger is sealed off by means of the seal (59). Inside the upper housing part is the stop (60) on which the power takeoff flange abuts when the spring is relaxed. On the power takeoff flange is the stop (61) on which the power takeoff flange abuts when the spring is biased. After the biasing of the spring the locking member (62) moves between the stop (61) and a support (63) in the upper housing part. The actuating button (64) is connected to the locking member. The upper housing part ends in the mouthpiece (65) and is sealed off by means of the protective cover (66) which can be placed thereon. The spring housing (67) with compression spring (68) is rotatably mounted on the upper housing part by means of the snap-in lugs (69) and rotary bearing. The lower housing part (70) is pushed over the spring housing. Inside the spring housing is the exchangeable storage container (71) for the fluid (72) which is to be atomized. The storage container is sealed off by the stopper (73) through which the hollow plunger projects into the storage container and is immersed at its end in the fluid (supply of active substance solution). The spindle (74) for the mechanical counter is mounted in the covering of the spring housing. At the end of the spindle facing the upper housing part is the drive pinion (75). The slider (76) sits on the spindle.

The nebulizer described above is suitable for nebulizing the aerosol preparations according to the invention to produce an aerosol suitable for inhalation. If the formulation according to the invention is nebulized using the method described above (RESPIMAT®) the quantity delivered should correspond to a defined quantity with a tolerance of not more than 25%, preferably 20% of this amount in at least 97%, preferably at least 98% of all operations of the inhaler (spray actuations). Preferably, between 5 and 30 mg of formulation, most preferably between 5 and 20 mg of formulation are delivered as a defmed mass on each actuation. However, the formulation according to the invention may also be nebulized by means of inhalers other than those described above, e.g., jet stream inhalers.

Accordingly, in a further aspect, the invention relates to pharmaceutical formulations in the form of propellant-free inhalable solutions or suspensions as described above combined with a device suitable for administering these formulations, preferably in conjunction with the RESPIMAT® device. Preferably, the invention relates to propellant-free inhalable solutions or suspensions characterized by the combination of active substances 1, 2, and 3 according to the invention in conjunction with the device known by the name RESPIMAT®. In addition, the present invention relates to the above-mentioned devices for inhalation, preferably the RESPIMAT® device, characterized in that they contain the propellant-free inhalable solutions or suspensions according to the invention as described hereinbefore.

The propellant-free inhalable solutions or suspensions according to the invention may take the form of concentrates or sterile inhalable solutions or suspensions ready for use, as well as the abovementioned solutions and suspensions designed for use in a RESPIMAT® device. Formulations ready for use may be produced from the concentrates, for example, by the addition of isotonic saline solutions. Sterile formulations ready for use may be administered using energy-operated fixed or portable nebulizers which produce inhalable aerosols by means of ultrasound or compressed air by the Venturi principle or other principles.

Accordingly, in another aspect, the present invention relates to pharmaceutical compositions in the form of propellant-free inhalable solutions or suspensions as described hereinbefore which take the form of concentrates or sterile formulations ready for use, combined with a device suitable for administering these solutions, characterized in that the device is an energy-operated free-standing or portable nebulizer which produces inhalable aerosols by means of ultrasound or compressed air by the Venturi principle or other methods.

The Examples which follow serve to illustrate the present invention in more detail without restricting the scope of the invention to the following embodiments by way of example.

### FORMULATION EXAMPLES

A. Inhalable Powders

The following embodiments relate to inhalable powders which contain lactose as excipient. The embodiments described by way of example contain the amounts of 1 (in the form of the bromide), 2 and 3 specified in the following table per 5 mg of these formulations. The amounts given for ingredient 3 are based on the free bases. From these, the skilled person will readily be able to calculate the amounts of corresponding acid addition salts.

| Example | μg of 1 | μg of 2 | μg of 3 |
|---|---|---|---|
| 1 | 60 | 200 μg of 2.4 | 60 μg of 3.7 |
| 2 | 60 | 200 μg of 2.5 | 60 μg of 3.7 |
| 3 | 60 | 200 μg of 2.6 | 60 μg of 3.7 |
| 4 | 60 | 200 μg of 2.7 | 60 μg of 3.7 |
| 5 | 60 | 200 μg of 2.4 | 60 μg of 3.8 |
| 6 | 60 | 200 μg of 2.5 | 60 μg of 3.8 |
| 7 | 60 | 200 μg of 2.6 | 60 μg of 3.8 |
| 8 | 60 | 200 μg of 2.7 | 60 μg of 3.8 |
| 9 | 60 | 200 μg of 2.4 | 60 μg of 3.33 |
| 10 | 60 | 200 μg of 2.5 | 60 μg of 3.33 |
| 11 | 60 | 200 μg of 2.6 | 60 μg of 3.33 |
| 12 | 60 | 200 μg of 2.7 | 60 μg of 3.33 |
| 13 | 60 | 200 μg of 2.4 | 60 μg of 3.34 |
| 14 | 60 | 200 μg of 2.5 | 60 μg of 3.34 |
| 15 | 60 | 200 μg of 2.6 | 60 μg of 3.34 |
| 16 | 60 | 200 μg of 2.7 | 60 μg of 3.34 |
| 17 | 60 | 200 μg of 2.4 | 60 μg of 3.45 |
| 18 | 60 | 200 μg of 2.5 | 60 μg of 3.45 |
| 19 | 60 | 200 μg of 2.6 | 60 μg of 3.45 |
| 20 | 60 | 200 μg of 2.7 | 60 μg of 3.45 |
| 21 | 60 | 125 μg of 2.4 | 80 μg of 3.7 |
| 22 | 60 | 125 μg of 2.5 | 80 μg of 3.7 |
| 23 | 60 | 125 μg of 2.6 | 80 μg of 3.7 |
| 24 | 60 | 125 μg of 2.7 | 80 μg of 3.7 |
| 25 | 60 | 125 μg of 2.4 | 80 μg of 3.8 |
| 26 | 60 | 125 μg of 2.5 | 80 μg of 3.8 |
| 27 | 60 | 125 μg of 2.6 | 80 μg of 3.8 |
| 28 | 60 | 125 μg of 2.7 | 80 μg of 3.8 |
| 29 | 60 | 125 μg of 2.4 | 80 μg of 3.33 |
| 30 | 60 | 125 μg of 2.5 | 80 μg of 3.33 |
| 31 | 60 | 125 μg of 2.6 | 80 μg of 3.33 |
| 32 | 60 | 125 μg of 2.7 | 80 μg of 3.33 |
| 33 | 60 | 125 μg of 2.4 | 80 μg of 3.34 |
| 34 | 60 | 125 μg of 2.5 | 80 μg of 3.34 |
| 35 | 60 | 125 μg of 2.6 | 80 μg of 3.34 |
| 36 | 60 | 125 μg of 2.7 | 80 μg of 3.34 |
| 37 | 60 | 125 μg of 2.4 | 80 μg of 3.45 |
| 38 | 60 | 125 μg of 2.5 | 80 μg of 3.45 |
| 39 | 60 | 125 μg of 2.6 | 80 μg of 3.45 |
| 40 | 60 | 125 μg of 2.7 | 80 μg of 3.45 |
| 41 | 60 | 250 μg of 2.4 | 30 μg of 3.7 |
| 42 | 60 | 250 μg of 2.5 | 30 μg of 3.7 |
| 43 | 60 | 250 μg of 2.6 | 30 μg of 3.7 |
| 44 | 60 | 250 μg of 2.7 | 30 μg of 3.7 |
| 45 | 60 | 250 μg of 2.4 | 30 μg of 3.8 |
| 46 | 60 | 250 μg of 2.5 | 30 μg of 3.8 |
| 47 | 60 | 250 μg of 2.6 | 30 μg of 3.8 |
| 48 | 60 | 250 μg of 2.7 | 30 μg of 3.8 |
| 49 | 60 | 250 μg of 2.4 | 30 μg of 3.33 |
| 50 | 60 | 250 μg of 2.5 | 30 μg of 3.33 |
| 51 | 60 | 250 μg of 2.6 | 30 μg of 3.33 |
| 52 | 60 | 250 μg of 2.7 | 30 μg of 3.33 |
| 53 | 60 | 250 μg of 2.4 | 30 μg of 3.34 |
| 54 | 60 | 250 μg of 2.5 | 30 μg of 3.34 |
| 55 | 60 | 250 μg of 2.6 | 30 μg of 3.34 |
| 56 | 60 | 250 μg of 2.7 | 30 μg of 3.34 |
| 57 | 60 | 250 μg of 2.4 | 30 μg of 3.45 |
| 58 | 60 | 250 μg of 2.5 | 30 μg of 3.45 |
| 59 | 60 | 250 μg of 2.6 | 30 μg of 3.45 |
| 60 | 60 | 250 μg of 2.7 | 30 μg of 3.45 |
| 61 | 40 | 200 μg of 2.4 | 60 μg of 3.7 |
| 62 | 40 | 200 μg of 2.5 | 60 μg of 3.7 |
| 63 | 40 | 200 μg of 2.6 | 60 μg of 3.7 |
| 64 | 40 | 200 μg of 2.7 | 60 μg of 3.7 |
| 65 | 40 | 200 μg of 2.4 | 60 μg of 3.8 |
| 66 | 40 | 200 μg of 2.5 | 60 μg of 3.8 |
| 67 | 40 | 200 μg of 2.6 | 60 μg of 3.8 |
| 68 | 40 | 200 μg of 2.7 | 60 μg of 3.8 |
| 69 | 40 | 200 μg of 2.4 | 60 μg of 3.33 |
| 70 | 40 | 200 μg of 2.5 | 60 μg of 3.33 |
| 71 | 40 | 200 μg of 2.6 | 60 μg of 3.33 |
| 72 | 40 | 200 μg of 2.7 | 60 μg of 3.33 |
| 73 | 40 | 200 μg of 2.4 | 60 μg of 3.34 |
| 74 | 40 | 200 μg of 2.5 | 60 μg of 3.34 |
| 75 | 40 | 200 μg of 2.6 | 60 μg of 3.34 |
| 76 | 40 | 200 μg of 2.7 | 60 μg of 3.34 |
| 77 | 40 | 200 μg of 2.4 | 60 μg of 3.45 |
| 78 | 40 | 200 μg of 2.5 | 60 μg of 3.45 |
| 79 | 40 | 200 μg of 2.6 | 60 μg of 3.45 |
| 80 | 40 | 200 μg of 2.7 | 60 μg of 3.45 |
| 81 | 40 | 125 μg of 2.4 | 80 μg of 3.7 |
| 82 | 40 | 125 μg of 2.5 | 80 μg of 3.7 |
| 83 | 40 | 125 μg of 2.6 | 80 μg of 3.7 |
| 84 | 40 | 125 μg of 2.7 | 80 μg of 3.7 |
| 85 | 40 | 125 μg of 2.4 | 80 μg of 3.8 |
| 86 | 40 | 125 μg of 2.5 | 80 μg of 3.8 |
| 87 | 40 | 125 μg of 2.6 | 80 μg of 3.8 |
| 88 | 40 | 125 μg of 2.7 | 80 μg of 3.8 |
| 89 | 40 | 125 μg of 2.4 | 80 μg of 3.33 |
| 90 | 40 | 125 μg of 2.5 | 80 μg of 3.33 |
| 91 | 40 | 125 μg of 2.6 | 80 μg of 3.33 |
| 92 | 40 | 125 μg of 2.7 | 80 μg of 3.33 |
| 93 | 40 | 125 μg of 2.4 | 80 μg of 3.34 |
| 94 | 40 | 125 μg of 2.5 | 80 μg of 3.34 |
| 95 | 40 | 125 μg of 2.6 | 80 μg of 3.34 |
| 96 | 40 | 125 μg of 2.7 | 80 μg of 3.34 |
| 97 | 40 | 125 μg of 2.4 | 80 μg of 3.45 |
| 98 | 40 | 125 μg of 2.5 | 80 μg of 3.45 |
| 99 | 40 | 125 μg of 2.6 | 80 μg of 3.45 |
| 100 | 40 | 125 μg of 2.7 | 80 μg of 3.45 |
| 101 | 40 | 250 μg of 2.4 | 30 μg of 3.7 |
| 102 | 40 | 250 μg of 2.5 | 30 μg of 3.7 |
| 103 | 40 | 250 μg of 2.6 | 30 μg of 3.7 |
| 104 | 40 | 250 μg of 2.7 | 30 μg of 3.7 |
| 105 | 40 | 250 μg of 2.4 | 30 μg of 3.8 |
| 106 | 40 | 250 μg of 2.5 | 30 μg of 3.8 |
| 107 | 40 | 250 μg of 2.6 | 30 μg of 3.8 |
| 108 | 40 | 250 μg of 2.7 | 30 μg of 3.8 |
| 109 | 40 | 250 μg of 2.4 | 30 μg of 3.33 |
| 110 | 40 | 250 μg of 2.5 | 30 μg of 3.33 |
| 111 | 40 | 250 μg of 2.6 | 30 μg of 3.33 |
| 112 | 40 | 250 μg of 2.7 | 30 μg of 3.33 |
| 113 | 40 | 250 μg of 2.4 | 30 μg of 3.34 |
| 114 | 40 | 250 μg of 2.5 | 30 μg of 3.34 |
| 115 | 40 | 250 μg of 2.6 | 30 μg of 3.34 |
| 116 | 40 | 250 μg of 2.7 | 30 μg of 3.34 |
| 117 | 40 | 250 μg of 2.4 | 30 μg of 3.45 |
| 118 | 40 | 250 μg of 2.5 | 30 μg of 3.45 |
| 119 | 40 | 250 μg of 2.6 | 30 μg of 3.45 |
| 120 | 40 | 250 μg of 2.7 | 30 μg of 3.45 |
| 121 | 80 | 200 μg of 2.4 | 60 μg of 3.7 |
| 122 | 80 | 200 μg of 2.5 | 60 μg of 3.7 |
| 123 | 80 | 200 μg of 2.6 | 60 μg of 3.7 |
| 124 | 80 | 200 μg of 2.7 | 60 μg of 3.7 |
| 125 | 80 | 200 μg of 2.4 | 60 μg of 3.8 |
| 126 | 80 | 200 μg of 2.5 | 60 μg of 3.8 |
| 127 | 80 | 200 μg of 2.6 | 60 μg of 3.8 |
| 128 | 80 | 200 μg of 2.7 | 60 μg of 3.8 |
| 129 | 80 | 200 μg of 2.4 | 60 μg of 3.33 |
| 130 | 80 | 200 μg of 2.5 | 60 μg of 3.33 |
| 131 | 80 | 200 μg of 2.6 | 60 μg of 3.33 |
| 132 | 80 | 200 μg of 2.7 | 60 μg of 3.33 |
| 133 | 80 | 200 μg of 2.4 | 60 μg of 3.34 |
| 134 | 80 | 200 μg of 2.5 | 60 μg of 3.34 |
| 135 | 80 | 200 μg of 2.6 | 60 μg of 3.34 |
| 136 | 80 | 200 μg of 2.7 | 60 μg of 3.34 |
| 137 | 80 | 200 μg of 2.4 | 60 μg of 3.45 |
| 138 | 80 | 200 μg of 2.5 | 60 μg of 3.45 |
| 139 | 80 | 200 μg of 2.6 | 60 μg of 3.45 |
| 140 | 80 | 200 μg of 2.7 | 60 μg of 3.45 |
| 141 | 80 | 125 μg of 2.4 | 80 μg of 3.7 |
| 142 | 80 | 125 μg of 2.5 | 80 μg of 3.7 |
| 143 | 80 | 125 μg of 2.6 | 80 μg of 3.7 |
| 144 | 80 | 125 μg of 2.7 | 80 μg of 3.7 |
| 145 | 80 | 125 μg of 2.4 | 80 μg of 3.8 |
| 146 | 80 | 125 μg of 2.5 | 80 μg of 3.8 |
| 147 | 80 | 125 μg of 2.6 | 80 μg of 3.8 |
| 148 | 80 | 125 μg of 2.7 | 80 μg of 3.8 |
| 149 | 80 | 125 μg of 2.4 | 80 μg of 3.33 |
| 150 | 80 | 125 μg of 2.5 | 80 μg of 3.33 |
| 151 | 80 | 125 μg of 2.6 | 80 μg of 3.33 |
| 152 | 80 | 125 μg of 2.7 | 80 μg of 3.33 |
| 153 | 80 | 125 μg of 2.4 | 80 μg of 3.34 |
| 154 | 80 | 125 μg of 2.5 | 80 μg of 3.34 |
| 155 | 80 | 125 μg of 2.6 | 80 μg of 3.34 |
| 156 | 80 | 125 μg of 2.7 | 80 μg of 3.34 |
| 157 | 80 | 125 μg of 2.4 | 80 μg of 3.45 |
| 158 | 80 | 125 μg of 2.5 | 80 μg of 3.45 |
| 159 | 80 | 125 μg of 2.6 | 80 μg of 3.45 |
| 160 | 80 | 125 μg of 2.7 | 80 μg of 3.45 |
| 161 | 80 | 250 μg of 2.4 | 30 μg of 3.7 |
| 162 | 80 | 250 μg of 2.5 | 30 μg of 3.7 |
| 163 | 80 | 250 μg of 2.6 | 30 μg of 3.7 |
| 164 | 80 | 250 μg of 2.7 | 30 μg of 3.7 |
| 165 | 80 | 250 μg of 2.4 | 30 μg of 3.8 |
| 166 | 80 | 250 μg of 2.5 | 30 μg of 3.8 |
| 167 | 80 | 250 μg of 2.6 | 30 μg of 3.8 |
| 168 | 80 | 250 μg of 2.7 | 30 μg of 3.8 |
| 169 | 80 | 250 μg of 2.4 | 30 μg of 3.33 |
| 170 | 80 | 250 μg of 2.5 | 30 μg of 3.33 |
| 171 | 80 | 250 μg of 2.6 | 30 μg of 3.33 |
| 172 | 80 | 250 μg of 2.7 | 30 μg of 3.33 |
| 173 | 80 | 250 μg of 2.4 | 30 μg of 3.34 |
| 174 | 80 | 250 μg of 2.5 | 30 μg of 3.34 |
| 175 | 80 | 250 μg of 2.6 | 30 μg of 3.34 |
| 176 | 80 | 250 μg of 2.7 | 30 μg of 3.34 |
| 177 | 80 | 250 μg of 2.4 | 30 μg of 3.45 |
| 178 | 80 | 250 μg of 2.5 | 30 μg of 3.45 |
| 179 | 80 | 250 μg of 2.6 | 30 μg of 3.45 |
| 180 | 80 | 250 μg of 2.7 | 30 μg of 3.45 |

Formulations analogous to the embodiments mentioned above may be obtained using the excipients trehalose or glucose, for example.

B. Propellant Gas-Containing Inhalable Aerosols

| | |
|---|---|
| Ingredients | Wt.-% |

1)

| | |
|---|---|
| 1′-bromide | 0.035 |
| budesonide | 0.4 |
| formoterol fumarate dihydrate | 0.066 |
| soya lecithin | 0.2 |
| TG134a:TG227 (2:3) | to 100 |

2)

| | |
|---|---|
| 1′-bromide | 0.039 |
| fluticasone-propionate | 0.3 |
| salmeterol xinafoate | 0.033 |
| isopropyl myristate | 0.1 |
| TG 227 | to 100 |

3)

| | |
|---|---|
| 1′-bromide | 0.039 |
| mometasone-furoate | 0.6 |
| salmeterol × diflunisal | 0.066 |
| isopropyl myristate | 0.1 |
| TG 227 | to 100 |

4)

| | |
|---|---|
| 1′-bromide | 0.035 |
| fluticasone-propionate | 0.3 |
| salmeterol-4-phenylcinnamate | 0.066 |
| soya lecithin | 0.2 |
| TG 11:TG12 (2:3) | to 100 |

5)

| | |
|---|---|
| 1′-bromide | 0.039 |
| salmeterol xinafoate | 0.033 |
| budesonide | 0.4 |
| absolute ethanol | 0.5 |
| isopropyl myristate | 0.1 |
| TG 227 | to 100 |

6)

| | |
|---|---|
| 1′-bromide | 0.039 |
| salmeterol xinafoate | 0.033 |
| ciclesonide | 0.4 |
| absolute ethanol | 0.5 |
| isopropyl myristate | 0.1 |
| TG 227 | to 100 |

7)

| | |
|---|---|
| 1′-bromide | 0.039 |
| mometasone-furoate | 0.6 |
| salmeterol xinafoate | 0.066 |
| isopropyl myristate | 0.1 |
| TG 227 | to 100 |

We Claim:

1. A pharmaceutical formulation comprising:
(a) at least one anticholinergic of formula 1 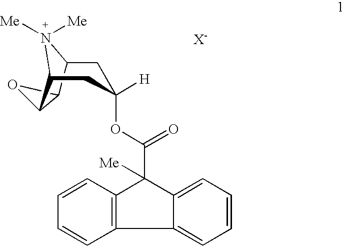
wherein X⁻ is an anion with a single negative charge;
(b) at least one corticosteroid (2); and
(c) at least one betamimetic (3),
and the enantiomers, mixtures of the enantiomers, racemates, solvates, hydrates, or physiologically acceptable acid addition salts thereof.

2. The pharmaceutical formulation according to claim 1, further comprising at least one pharmacologically acceptable excipient.

3. The pharmaceutical formulation according to claim 1, wherein X⁻ is chloride, bromide, iodide, sulfate, phosphate, methanesulfonate, nitrate, maleate, acetate, citrate, fumarate, tartrate, oxalate, succinate, benzoate, and p-toluenesulfonate.

4. The pharmaceutical formulation according to claim 1, wherein X⁻ is chloride, bromide, p-toluenesulfonate, or methanesulfonate.

5. The pharmaceutical formulation according to one of claims 1 to 4, wherein at least one corticosteroid is flunisolide, beclomethasone, triamcinolone, budesonide, fluticasone, mometasone, ciclesonide, rofleponide, GW 215864, KSR 592, ST-126, dexamethasone, etiprednol, loteprednol, EPI-177, and PLD-177, and the enantiomers, mixtures of the enantiomers, racemates, solvates, hydrates, or physiologically acceptable acid addition salts thereof.

6. The pharmaceutical formulation according to one of claims 1 to 4, wherein at least one betamimetic is albuterol, bambuterol, bitolterol, broxaterol, carbuterol, clenbuterol, fenoterol, formoterol, hexoprenaline, ibuterol, isoetharine, isoprenaline, levosalbutamol, mabuterol, meluadrine, metaproterenol, orciprenaline, pirbuterol, procaterol, reproterol, TD 3327, ritodrin, salmeterol, salmefamol, soterenot, sulfonterol, tiaramide, terbutaline, tolubuterol, CHF4226, HOKU-81, KUL-1248, 3-(4-{6-[2-hydroxy-2-(4-hydroxy-3-hydroxymethylphenyl)ethylamino]hexyloxy}butyl)benzenesulfonamide, 5-[2-(5,6-diethylindan-2-ylamino)-1-hydroxyethyl]-8-hydroxy-1H-quinolin-2-one, 4-hydroxy-7-[2-{[2-{[3-(2-phenylethoxy)propyl]sulfonyl}ethyl]amino}ethyl]-2(3H)-benzothiazolone, 1-(2-fluoro-4-hydroxyphenyl)-2-[4-(1-benzimidazolyl)-2-methyl-2-butylamino]ethanol, 1-[3-(4-methoxybenzylamino)-4-hydroxyphenyl]-2-[4-(1-benzimidazolyl)-2-methyl-2-butylamino]ethanol, 1-[2H-5-hydroxy-3-oxo-4H-1,4-benzoxazin-8-yl]-2-[3-(4-N,N-dimethylaminophenyl)-2-methyl-2-propylamino]ethanol, 1-[2H-5-hydroxy-3-oxo-4H- 1,4-benzoxazin-8-yl]-2-[3-(4-methoxyphenyl)-2-methyl-2-propylamino]ethanol, 1-[2H-5-hydroxy-3-oxo-4H-1,4-benzoxazin-8-yl]-2-[3-(4-n-butyloxyphenyl)-2-methyl-2-propylamino]ethanol, 1-[2H-5-hydroxy-3-oxo-4H-1,4-benzoxazin-8-yl]-2-{4-[3-(4-methoxyphenyl)-1,2,4-triazol-3-yl]-2-methyl-2-butylamino}ethanol, 5-hydroxy-8-(1-hydroxy-2-isopropylaminobutyl)-2H-1,4-benzoxazin-3-(4H)-one, 1-(4-amino-3-chloro-5-trifluoromethylphenyl)-2-tert-butylamino)ethanol, 1-(4-ethoxycarbonylamino-3-cyano-5-fluorophenyl)-2-(tert-butylamino)ethanol, and N-[2-hydroxy-5-(1-hydroxy-2-{2-[4-(2-hydroxy-2-phenylethylamino)phenyl]ethylamino}ethyl)phenyl]formamide, and the enantiomers, mixtures of the enantiomers, racemates, solvates, hydrates, or physiologically acceptable acid addition salts thereof.

7. The pharmaceutical formulation according to one of claims 1 to 4, wherein the weight ratio of 1′ to 2 is in the range from 1:250 to 250:1.

8. The pharmaceutical formulation according to one of claims 1 to 4, wherein the weight ratio of 1′ to 2 is in the range from 1:150 to 150:1.

9. The pharmaceutical formulation according to one of claims 1 to 4, wherein the weight ratio of 1 to 3 is in the range from 1:300 to 30:1.

10. The pharmaceutical formulation according to one of claims 1 to 4, wherein the weight ratio of 1 to 3 is in the range from 1:230 to 20:1.

11. The pharmaceutical formulation according to claim 1, wherein the pharmaceutical formulation is suitable for inhalation.

12. The pharmaceutical formulation according to claim 1, wherein the pharmaceutical formulation is an inhalable powder, propellant-containing metered-dose aerosol, or propellant-free inhalable solution or suspension.

13. The pharmaceutical formulation according to claim 2, wherein the pharmaceutical formulation is an inhalable powder, propellant-containing metered-dose aerosol, or propellant-free inhalable solution or suspension.

14. The pharmaceutical formulation according to claim 13, wherein at least one pharmacologically acceptable excipient is a monosaccharide, disaccharide, oligo- and polysaccharide, polyalcohol, salt, or a mixture thereof.

15. The pharmaceutical formulation according to claim 13, wherein the excipient has a maximum average particle size of up to 250 μm.

16. A capsule comprising the pharmaceutical formulation according to one of claims 12 to 15.

17. The pharmaceutical formulation according to claim 12, consisting essentially of:
(a) at least one anticholinergic of formula 1;
(b) at least one corticosteroid; and
(c) at least one betamimetic,
and the enantiomers, mixtures of the enantiomers, racemates, solvates, hydrates, or physiologically acceptable acid addition salts thereof.

18. The pharmaceutical formulation according to claim 12, wherein the pharmaceutical formulation is a propellant-containing inhalable aerosol which contains (a) at least one anticholinergic of formula 1, (b) at least one corticosteroid, and (c) at least one betamimetic in dissolved or dispersed form.

19. The pharmaceutical formulation according to claim 18, wherein the propellant gas is a hydrocarbon or halohydrocarbon.

20. The pharmaceutical formulation according to claim 18, wherein the propellant gas is n-propane, n-butane, or isobutane, or a chlorinated and/or fluorinated derivative of methane, ethane, propane, butane, cyclopropane, or cyclobutane, or a mixture thereof.

21. The pharmaceutical formulation according to claim 18, wherein the propellant gas is TG11, TG12, TG134a, TG227, or a mixture thereof.

22. The pharmaceutical formulation according to claim 18, wherein the pharmaceutical composition is a propellant-free inhalable solution containing water, ethanol, or a mixture thereof as solvent.

23. The pharmaceutical formulation according to claim 18, further comprising a cosolvent and/or excipient.
